(19) United States
(12) Patent Application Publication
(10) Pub. No.: US 2023/0064985 A1
(43) Pub. Date: Mar. 2, 2023

(54) SMART SCENT LIBRARY

(71) Applicant: Micron Technology, Inc., Boise, ID (US)

(72) Inventors: Trupti D. Gawai, Boise, ID (US); Brooke Spencer, Midvale, UT (US); Erica A. Ellingson, Boise, ID (US)

(21) Appl. No.: 17/464,016

(22) Filed: Sep. 1, 2021

(51) Int. Cl. G16H 10/65; G16C 20/40; G06Q 30/02

(52) U.S. Cl. CPC G16H 10/65; G16C 20/40; G06Q 30/02

(57) ABSTRACT

Methods, systems, and devices associated with a smart scent library are described. An apparatus can include a processing resource and a memory resource having instructions executable to store data representing a number of chemical compositions in a smart scent library. The instructions can be executable to receive data, including a chemical composition, and compare the chemical composition to the number of chemical compositions. The instructions can be executable to identify the chemical composition in response to matching at least a portion of the chemical composition to one of the number of chemical compositions, and transmit data associated with the identified chemical composition.

### TECHNICAL FIELD

The present disclosure relates generally to apparatuses, systems, and methods associated with a smart scent library.

### BACKGROUND

A computing device can be a smartphone, a wearable device, a tablet, a laptop, a desktop computer, a smart assistant device, or a cloud computing device, for example. The computing device can receive and/or transmit data and can include or be coupled to one or more memory devices. Memory devices are typically provided as internal, semiconductor, integrated circuits in computers or other electronic systems. There are many different types of memory including volatile and non-volatile memory. Volatile memory can require power to maintain its data (e.g., host data, error data, etc.) and includes random access memory (RAM), dynamic random-access memory (DRAM), static random-access memory (SRAM), synchronous dynamic random-access memory (SDRAM), and thyristor random access memory (TRAM), among others. Non-volatile memory can provide persistent data by retaining stored data when not powered and can include NAND flash memory, NOR flash memory, and resistance variable memory such as phase change random access memory (PCRAM), resistive random-access memory (RRAM), and magnetoresistive random access memory (MRAM), such as spin torque transfer random access memory (STT RAM), among others.

### BRIEF DESCRIPTION OF THE DRAWINGS

FIG. 1 illustrates an example of a computing device including a smart scent library for identifying a chemical composition in accordance with a number of embodiments of the present disclosure.

FIG. 2 illustrates an example of a computing device including a smart scent library for identifying a chemical composition in accordance with a number of embodiments of the present disclosure.

FIG. 3 illustrates an example of a flow diagram for identifying a chemical composition using a smart scent library in accordance with a number of embodiments of the present disclosure.

FIG. 4 is a flow diagram of a method for identifying a chemical composition using a smart scent library in accordance with a number of embodiments of the present disclosure.

### DETAILED DESCRIPTION

The present disclosure includes apparatuses and methods related to storing data representing a number of chemical compositions in a smart scent library, receiving data, including a chemical composition, comparing the chemical composition to the number of chemical compositions, identifying the chemical composition in response to matching at least a portion of the chemical composition to one of the number of chemical compositions, and transmit data associated with the identified chemical composition.

Often people smell scents (e.g., smells) that they cannot identify. For example, a person may not be able to determine what is producing a scent and/or what the scent is because they are unable to ask someone what the scent is and/or search the scent.

A scent can be identified using a smart scent library. The smart scent library can include data corresponding to a number of chemical compositions, a number of locations, a number of scent categories, a number of allergens, a number of dangerous chemicals, and/or a number of advertisements. In a number of embodiments, data including a chemical composition, a location, particle data, and/or a user input can be received by the smart scent library. The received data can be compared to the data included in the smart scent library to determine a chemical composition of the scent, a name of the scent, a category of the scent, a warning about the scent, an advertisement associated with the scent, and/or a location of the scent. In some examples, the chemical composition of the scent, the name of the scent, the category of the scent, the warning about the scent, the advertisement associated with the scent, and/or the location of the scent can be conveyed to a user using, for example, a user interface. The scent library can also receive user feedback to verify or modify the smart scent library.

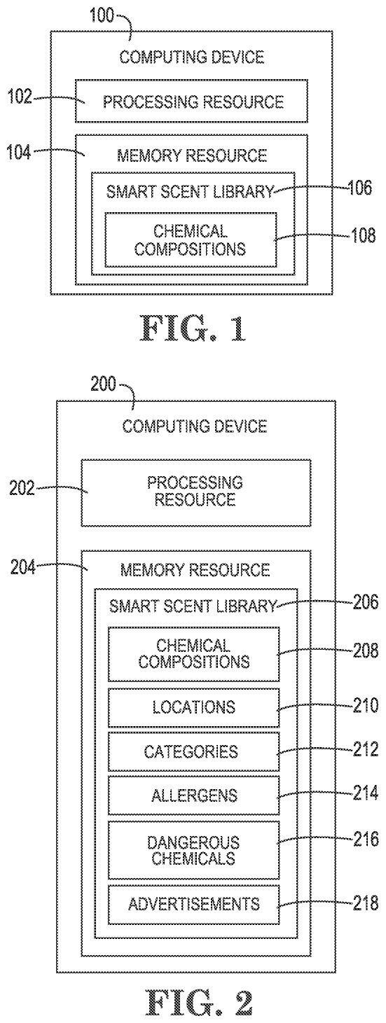

As used herein, “a number of” something can refer to one or more of such things. A “plurality” of something intends two or more. The figures herein follow a numbering convention in which the first digit or digits correspond to the drawing figure number and the remaining digits identify an element or component in the drawing. Similar elements or components between different figures may be identified by the use of similar digits. For example, reference numeral 102 may reference element “2” in FIG. 1, and a similar element may be referenced as 202 in FIG. 2. In some instances, a plurality of similar, but functionally and/or structurally distinguishable, elements or components in the same figure or in different figures may be referenced sequentially with the same element number. As will be appreciated, elements shown in the various embodiments herein can be added, exchanged, and/or eliminated so as to provide a number of additional embodiments of the present disclosure. In addition, the proportion and the relative scale of the elements provided in the figures are intended to illustrate various embodiments of the present disclosure and are not to be used in a limiting sense.

FIG. 1 illustrates an example of a computing device 100 including a smart scent library 106 for identifying a chemical composition in accordance with a number of embodiments of the present disclosure. The computing device 100 can be, but is not limited to, a cloud computing device, a smartphone, a wearable device, a tablet, a laptop, a desktop computer, a smart assistant device, or any combination thereof.

A processing resource 102 coupled to a memory resource 104 can be included in and/or coupled to the computing device 100. The computing device 100 can receive and/or transmit data via wired and/or wireless transmissions using a communication device (e.g., intermediary device), such as, but not limited to a radio, not illustrated. The radio through signaling (e.g., radio signals) and/or a network relationship can enable the computing device 100 to communicate with one or more components of the computing device 100, one or more other computing devices, wearable devices, telephones, sensors, smart assistants, and/or cloud computing devices. Examples of such a network relationship can include Bluetooth, AirDrop, a peer-to-peer Wi-Fi network, a cellular network, a distributed computing environment (e.g., a cloud computing environment), a wide area network (WAN) such as the Internet, a local area network (LAN), a personal area network (PAN), a campus area network (CAN), or metropolitan area network (MAN), among other types of network relationships.

The memory resource (e.g., memory) 104 can include volatile and/or non-volatile memory, for instance, DRAM, NAND, and/or 3D Cross-point. The memory 104 can be coupled to the processing resource 102 and can store a smart scent library 106 including chemical compositions 108. The memory resource 104 can be any type of storage medium that can be accessed by the processing resource 102 to perform various examples of the present disclosure. For example, the memory 104 can be a non-transitory computer readable medium having computer readable instructions (e.g., computer program instructions) stored thereon that are executable by the processing resource 102 to store, in the memory resource 104, data representing the number of chemical compositions 108 in the smart scent library 106, receive data, including a chemical composition, compare the chemical composition to the number of chemical compositions 108, identify the chemical composition in response to matching at least a portion of the chemical composition to one of the number of chemical compositions 108, and transmit data associated with the identified chemical composition. In some examples, the computing device 100 can receive the data from a different computing device and/or transmit the data associated with the identified chemical composition to the different computing device.

FIG. 2 illustrates an example of a computing device 200 including a smart scent library 206 for identifying a chemical composition in accordance with a number of embodiments of the present disclosure. Computing device 200, processing resource 202, memory resource 204, and chemical compositions 208 can correspond to computing device 100, processing resource 102, memory resource 104, and chemical compositions 108, respectively of FIG. 1. The smart scent library 206 can further include a number of locations 210, a number of categories 212, a number of allergens 214, a number of dangerous chemicals 216, and/or a number of advertisements 218.

In some examples, the memory resource 204 can further include computer readable instructions stored thereon that are executable by the processing resource 202 to store, in the memory resource 204, the number of locations 210 and data representing the number of chemical compositions 208 corresponding to each of the number of locations 210 in the smart scent library 206, receive data, including a location, compare the location to the number of locations 210, identify a chemical composition associated with the location in response to one of the number of locations 210 being within a particular proximity of the location, and transmit data associated with the identified chemical composition. The received data can also include a user input, which can be a filter. The filter can be used to filter out one or more of the number of chemical compositions 208 based on the filter prior to identifying the chemical composition associated with the location. For example, a user can convey that they are indoors, accordingly, chemical compositions produced outdoors can be filtered out of the number of chemical compositions 208. This can improve speed and accuracy of the identification of the chemical composition.

The memory resource 204 can include computer readable instructions stored thereon that are executable by the processing resource 202 to determine whether an identified chemical composition is included in a particular category of the number of categories 212 and transmit the data associated with the identified chemical composition in response to determining the identified chemical composition is included in the particular category. A user may have access to some categories, but not others. For example, a user may have access to environmental scents, but not industrial scents. As such, a user may receive an error message and/or an option to purchase access to industrial scents in response to transmitting a chemical composition that the smart scent library 206 identifies as an industrial scent.

In a number of embodiments, the memory resource 204 can further include computer readable instructions stored thereon that are executable by the processing resource 202 to compare the identified chemical composition to a number of allergens 214 of a user. The user can input the number of allergens 214 into the smart scent library 206. When the identified chemical composition matches one of the number of allergens 214, the transmitted data associated with the identified chemical composition can include a warning indicating the identified chemical composition is one of the number of allergens 214.

The transmitted data associated with the identified chemical composition can also include a warning when the identified chemical composition is a dangerous chemical composition. A dangerous chemical composition can be a chemical that can cause harm to a person and/or an animal from inhaling the chemical composition. The memory resource 204 can include computer readable instructions stored thereon that are executable by the processing resource 202 to compare the identified chemical composition to a number of dangerous chemical compositions (e.g., dangerous chemicals) 216. When the identified chemical composition matches one of the number of dangerous chemical compositions 216, the transmitted data associated with the identified chemical composition can include a warning indicating the identified chemical composition is dangerous.

In some examples, the memory resource 204 can include computer readable instructions stored thereon that are executable by the processing resource 202 to compare the identified chemical composition to a number of chemical compositions 208 each corresponding to an advertisement of a number of advertisements 218. When the identified chemical composition matches one of the number of chemical compositions 208 corresponding to an advertisement, the transmitted data associated with the identified chemical composition can include the advertisement. For example, the identified chemical composition can be from a pretzel sold in a shopping mall. The advertisement can include a name of the kiosk selling the pretzels, a location of the kiosk, nutritional information of the pretzels, customer reviews of the pretzels, a price of the pretzels, and/or a coupon for buying a pretzel. Another example can include identifying the chemical composition as a perfume. The advertisement can include an Internet link to purchasing the perfume online or provide perfumes with similar chemical compositions to the identified chemical composition based on a user's preferences that may be cheaper or produced without animal testing, for example.

Advertisements 218 can also be included in the transmitted data associated with the identified chemical composition in response to the identified chemical composition being associated with a location. The memory resource 204 can include computer readable instructions stored thereon that are executable by the processing resource 202 to compare the location to a number of locations 210 corresponding to an advertisement. When the location matches one of the number of locations 210 corresponding to an advertisement, the transmitted data associated with the identified chemical composition can include the advertisement. For example, the advertisement can be for a mask if the location is known for poor air quality or the advertisement can be for a restaurant nearby.

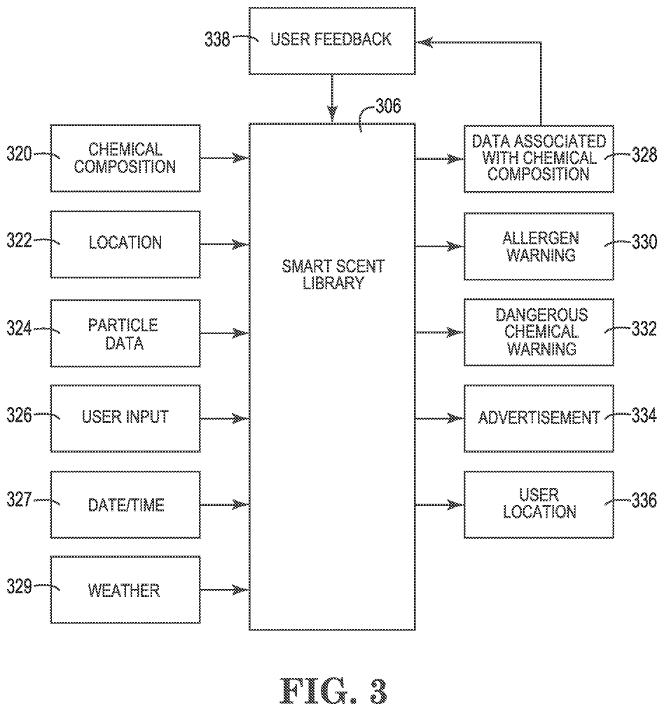

FIG. 3 illustrates an example of a flow diagram for identifying a chemical composition using a smart scent library 306 in accordance with a number of embodiments of the present disclosure. Smart scent library 306 can correspond to smart scent library 106 and smart scent library 206 in FIG. 1 and FIG. 2, respectively. The smart scent library 306 can receive data including a chemical composition 320, a location 322, particle data 324, a user input 326, a date and/or time 327, weather data 329, and/or user feedback 338. In some examples, the smart scent library 306 can output data including data associated with a chemical composition 328, an allergen warning 330, a dangerous chemical warning 332, an advertisement 334, and/or a user location 336 in response to receiving data.

The chemical composition data 320 can include a number of chemical elements that make up a particular compound. For example, a chemical sensor and/or a gas detector can identify a chemical composition including nitrocellulose and ethyl acetate. The smart scent library 306 can compare this chemical composition to a number of chemical compositions. The chemical composition can match with one of the number of chemical compositions that is a nail polish, for instance. Accordingly, the data associated with the chemical composition 328 can include identifying the chemical composition 320 as a nail polish.

The location 322 can be received by the smart scent library 306 as coordinates and/or an address, for example. The location 322 can be determined via a global positioning system (GPS), a location tag on social media, and/or provided by a user. The location 322 can be used by the smart scent library 306 to identify scents previously identified in that location with and/or without receiving a chemical composition 320 along with the location 322. For example, coordinates of the location 322 may place the user at a beach on the ocean, accordingly the smart scent library 306 can identify the chemical composition as sea air.

In a number of embodiments, the location 322 can be used in an internet query. For example, the location 322 can be a field that is used as fair grounds on particular days of the year. The internet query including the location 322 and/or a date and/or time 327 can determine a category of scents that are different depending on the date and/or time 327. For example, on most days the scents from the field can be from grasses and/or flowers and on fair days the scents from the field can be from fair foods and/or animals exhibited at the fair.

The particle data 324 can be received by the smart scent library 306 as a quantity of particles and/or a size of particles. The particle data 324 can be determined using a particle counter, for example. The smart scent library 306 can use the particle data 324 to identify a chemical composition and the concentration of the scent by comparing the particle data 324 to data included in the smart scent library 306 and identifying a scent in response to matching the particle data to a portion of the data included in the smart scent library 306. For example, the smart scent library 306 can identify a scent as mold and/or a specific type of mold based on its particle size and the concentration of the mold based on the quantity of particles. The smart scent library 306 can determine the mold is an allergen and transmit the allergen warning 330 based on identifying the scent as mold and/or a specific type of mold. A dangerous chemical warning 332 can be transmitted in response to determining the mold is dangerous based on the type of mold and/or the concentration of the mold.

The user input 326 can be received by the smart scent library 306 as text data or audio data from a user interface of a computing device, for example. The user input 326 can identify a scent and/or information associated with a scent to add a scent and/or scent information to the smart scent library 306. In some examples, the user input 326, as previously discussed in connection with FIG. 2, can be used as a filter. The received data associated with user input 326 can be compared to data included in the smart scent library 306. The received data associated with user input 326 can be compared to a portion of the data included in the smart scent library 306 in response to filtering out some of the data included in the smart scent library 306 based on the filter. A scent can be identified in response to matching the data associated with the user input 326 to a portion of the data included in the smart scent library 306.

A date and/or time 327 can be received by the smart scent library 306. The date and/or time 327 can be a timestamp from a computing device, for example. The date and/or time 327 can be used to determine a scent. For example, the smart scent library 306 can determine which flowers may be providing a scent due to them flowering on particular dates and/or at particular times.

Weather data 329 can be received by the smart scent library 306. The weather data 329 can include past, present, and/or future weather data. In a number of embodiments, the weather data 329 can be based on the location 322 input. For example, if the weather data 329 includes local weather reporting a forest fire, the smart scent library 306 can increase its confidence that the scent is smoke.

User feedback 338 can be received by the smart scent library 306 after the smart scent library 306 has transmitted an output, for example, the data associated with the chemical composition 328. The data associated with the chemical composition 328 can include a determined confidence level for a number of identified scents. A user may select one of the number of identified scents as the scent the user believes is the scent. The smart scent library 306 can revise the data included in the smart scent library 306 in response to receiving the selection of the one or the number of scents, which could change a future output and/or confidence level of the smart scent library 306. For example, if the user selected the scent the smart scent library 306 provided with the highest confidence level, the smart scent library 306 can increase the confidence level for the identified scent even higher. If the user selected the scent the smart scent library 306 provided with the lowest confidence level, the smart scent library 306 can decrease the confidence level for the wrongly identified scents and increase the confidence level for the selected scent.

In a number of embodiments, a smart scent library 306 can identify a user location 336 based on the received chemical composition 320. The received chemical composition 320 can include a unique number of scents only found in a particular location. For example, the smart scent library 306 can identify a first scent from a particular type of tree and a second scent from a particular type of flower and identify areas where both live. The smart scent library can further use seasons, for example, to further narrow down an area where that particular flower is blooming.

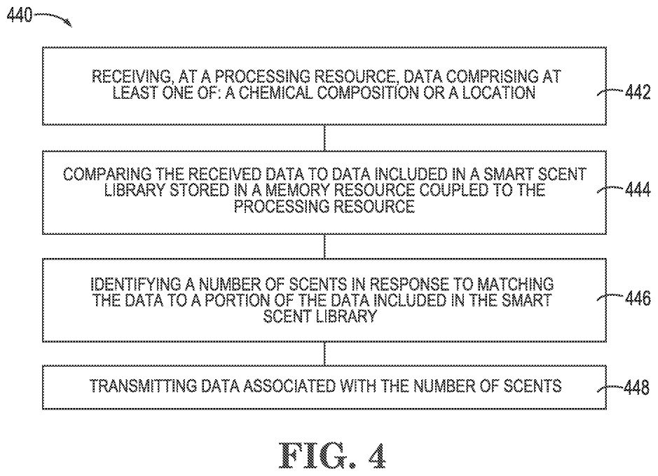

FIG. 4 is a flow diagram of a method 440 for identifying a chemical composition using a smart scent library in accordance with a number of embodiments of the present disclosure. At block 442, the method 440 can include receiving, at a processing resource, data comprising at least one of: a chemical composition or a location.

At block 444, the method 440 can include comparing the received data to data included in a smart scent library stored in a memory resource coupled to the processing resource. The received data can include a chemical composition, a location, particle data, a user input, and/or user feedback.

At block 446, the method 440 can include identifying a number of scents in response to matching the data to a portion of the data included in the smart scent library. The data included in the smart scent library can be chemical compositions, locations, categories, allergens, dangerous chemicals, and/or advertisements.

At block 448, the method 440 can include transmitting data associated with the number of scents. The data associated with the number of scents can include a scent identification, an allergen warning, a dangerous chemical warning, and/or an advertisement.

In a number of embodiments, the method 440 can further include determining a confidence level for each of the number of identified scents. The confidence level can be based on the amount of data that matches the portion of the data included in the smart scent library. For example, if most of the data matches the portion of the data included in the smart scent library, a high confidence level can be determined and if less of the data matches the portion of the data included in the smart scent library, a lower confidence level can be determined. The determined confidence level can be transmitted and conveyed to a user.

The method 440 can further include receiving a selection of one of the number of scents and revising the data included in the smart scent library in response to receiving the selection of the one of the number of scents. For example, a user can provide user feedback by selecting and/or inputting one of the number of scents identified by the smart scent library that the user believes is the likely scent. Accordingly, the smart scent library can revise the data included in the smart scent library based on the user's selection and/or input.

In some examples, the method 440 can further include receiving, at the processing resource, particle data, comparing the particle data to the data included in the smart scent library, and identifying the number of scents in response to matching the particle data to the portion of the data included in the smart scent library. The particle data can be received by the smart scent library as a quantity of particles and/or a size of particles and the smart scent library can identify the number of scents based on the quantity of particles and/or the size of particles. For example, each of the number of scents stored in the smart scent library can include a quantity of particles and/or a size of particles and the smart scent library can match the received quantity of particles and/or size of particles to one or more of the number of scents with a close quantity of particles and/or a close size of particles.

The method 440 can further include receiving, at the processing resource, data associated with a user input, comparing the data associated with the user input to data included in the smart scent library, and identifying the number of scents in response to matching the data associated with the user input to the portion of the data included in the smart scent library. The user input can be received via a user interface of a computing device as text data or audio data. In some examples, the user input can identify a scent and/or provide information associated with a scent. The user input can also be used as a filter to filter out some of the data included in the smart scent library.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An apparatus, comprising:
a processing resource; and
a memory resource in communication with the processing resource having instructions executable to:
store, in the memory resource, data representing a number of chemical compositions in a smart scent library;
receive data including a chemical composition;
compare the chemical composition to the number of chemical compositions;
identify the chemical composition in response to matching at least a portion of the chemical composition to one of the number of chemical compositions; and
transmit data associated with the identified chemical composition.

2. The apparatus of claim 1, further comprising the memory resource having instructions executable to:
determine whether the identified chemical composition is included in a particular category; and
transmit the data associated with the identified chemical composition in response to determining the identified chemical composition is included in the particular category.

3. The apparatus of claim 1, wherein the data associated with the identified chemical composition includes a warning regarding the identified chemical composition.

4. The apparatus of claim 3, further comprising the memory resource having instructions executable to compare the identified chemical composition to a number of allergens of a user, wherein the data associated with the identified composition includes the warning regarding the identified chemical composition in response to matching the identified chemical composition to one of the number of allergens of the user.

5. The apparatus of claim 3, further comprising the memory resource having instructions executable to compare the identified chemical composition to a number of dangerous chemical compositions, wherein the data associated with the identified composition includes the warning regarding the identified chemical composition in response to matching the identified chemical composition to one of the number of dangerous chemical compositions.

6. The apparatus of claim 1, wherein the data associated with the identified chemical composition includes an advertisement associated with the identified chemical composition.

7. The apparatus of claim 6, further comprising the memory resource having instructions executable to compare the identified chemical composition to a number of chemical compositions corresponding to an advertisement, wherein the data associated with the identified chemical composition includes the advertisement in response to matching the identified chemical composition to one of the number of chemical compositions corresponding to the advertisement.

8. An apparatus, comprising:
a processing resource; and
a memory resource in communication with the processing resource having instructions executable to:
store, in the memory resource, a number of locations and data representing a number of chemical compositions corresponding to each of the number locations in a smart scent library;
receive data, including a location;
compare the location to the number of locations;
identify a chemical composition associated with the location in response to one of the number of locations being within a particular proximity of the location; and
transmit data associated with the identified chemical composition.

9. The apparatus of claim 8, wherein the received data includes the location and a user input.

10. The apparatus of claim 9, wherein the user input is a filter.

11. The apparatus of claim 10, further comprising the memory resource having instructions executable to filter out one or more of the number of chemical compositions based on the filter prior to identifying the chemical composition associated with the location.

12. The apparatus of claim 8, wherein the data associated with the identified chemical composition includes an advertisement associated with the location.

13. The apparatus of claim 12, further comprising the memory resource having the instructions executable to compare the location to a number of locations corresponding to an advertisement, wherein the data associated with the identified composition includes the advertisement in response to matching the location to one of the number of locations corresponding to the advertisement.

14. A method, comprising:
receiving, at a processing resource, data comprising at least one of: a particle count, a chemical composition, or a location;
comparing the data to data included in a smart scent library stored in a memory resource coupled to the processing resource;
identifying a number of scents in response to matching the received data to a portion of the data included in the smart scent library; and
transmitting data associated with the number of scents.

15. The method of claim 14, further comprising:
determining a confidence level for each of the number of scents; and
transmit the confidence level for each of the number of scents.

16. The method of claim 14, further comprising:
receiving a selection of one of the number of scents; and
revising the data included in the smart scent library in response to receiving the selection of the one of the number of scents.

17. The method of claim 14, further comprising:
receiving, at the processing resource, particle data;
comparing the particle data to the data included in the smart scent library; and
identifying the number of scents in response to matching the particle data to the portion of the data included in the smart scent library.

18. The method of claim 14, further comprising:
receiving, at the processing resource, data associated with a user input;
comparing the data associated with the user input to data included in the smart scent library; and
identifying the number of scents in response to matching the data associated with the user input to the portion of the data included in the smart scent library.

19. The method of claim 18, further comprising receiving the data associated with the user input as at least one of: text data or audio data.

20. The method of claim 18, further comprising receiving, at the processing resource, the data associated with the user input via a user interface of a computing device.
